(19) United States
(12) Patent Application Publication
(10) Pub. No.: US 2007/0032755 A1
(43) Pub. Date: Feb. 8, 2007

(54) Method and apparatus for treating a wound

(75) Inventor: Richard F. Walsh, Fort Worth, TX (US)

Correspondence Address: BRACEWELL & GIULIANI LLP, P.O. BOX 61389, HOUSTON, TX, 77208-1389, US

(73) Assignee: MEDICA-RENTS CO., LTD.

(21) Appl. No.: 11/483,155

(22) Filed: Jul. 7, 2006

Related U.S. Application Data
(63) Continuation-in-part of application No. 11/194,970, filed on Aug. 2, 2005.

(51) Int. Cl. A61F 5/00

(52) U.S. Cl. 602/2

(57) ABSTRACT

The present invention provides for a method and apparatus for treating a wound. In a preferred method, collagen is applied to a wound; a drain tube is placed adjacent the wound, and a wound dressing is applied over the collagen and the drain tube. The preferred method further includes the step of draining fluids from the wound through the drain tube, which tube has a proximal end positioned adjacent the wound and a distal end in fluid communication with a vacuum source. The fluids are drained from the wound by applying a negative pressure to the wound through the tube from the vacuum source.

### RELATED APPLICATION

This application is a continuation-in-part of and claims benefit and priority to U.S. patent application Ser. No. 11/194,970, filed on Aug. 2, 2005, which is incorporated herein by reference in its entirety.

### FIELD OF THE INVENTION

The present invention relates generally to a method and apparatus for treating a wound, and more particularly to the application of negative pressure to a wound treated with collagen.

### BACKGROUND OF THE INVENTION

Wound dressings typically are applied over various types of wounds to promote healing and to reduce the risk of infection. Although various types of dressing materials have been successfully employed, wound dressings comprising semi-permeable materials often are preferred because they can increase patient comfort and lower the risk of infection. Semi-permeable wound dressings generally pass moisture vapors, but generally are impervious or impermeable to liquids. Thus, they can promote healing by permitting a wound site to breathe.

Problems can arise with semi-permeable wound dressings when they are placed over draining wounds because they tend to retain fluid. For example, surgical wounds often tend to drain for a post-operative period of about forty-eight hours. The fluid that can accumulate under a semi-permeable wound dressing during a draining period can macerate the underlying tissue, cause infection, and otherwise inhibit healing. A procedure for alleviating this problem involves periodically piercing the wound dressing, draining the accumulated fluids, and resealing the wound dressing opening. However, such a procedure is time-consuming for health care professionals and, unless it is conducted at relatively frequent intervals, can be relatively ineffective in dealing with the problems associated with trapped fluid accumulation. Other procedures which involve opening or changing wound dressings tend to have problems associated with exposing a wound to a greater risk of infection and can be uncomfortable for patients.

Collagen is a natural biomaterial and is understood to be particularly useful for wound healing. Collagen plays an integral role during each phase of wound healing and is an excellent hemostatic agent. It also can absorb 40 to 60 times its weight in fluid. Furthermore, the fatty acids found in collagen have antimicrobial properties.

As understood by those skilled in the art, information on the structure and function of collagen has been derived largely from studies on selected higher vertebrate species, including humans. Collagen is a prevalent protein found in various types of animals, including humans, as understood by those skilled in the art. Chains of amino acids make up collagen, which is a constituent of the extracellular matrices and connective tissues from a wide variety of multicellular organisms. In these organisms, aggregates of collagen molecules coursing through tissues are responsible for establishing and maintaining the physical integrity of diverse extracellular structures, thereby contributing to the functional capabilities of the organism as a whole.

At the molecular level, collagen is a protein containing lengthy domains of triple-helical conformation, as understood by those skilled in the art. The unique collagen fold is made possible by virtue of the repetitive Gly-X-Y sequences in participating chains. In this type of sequence, glycine occurs in every third position along the chain. Additionally, the collagen protein participates in the formation of extracellular aggregates that function primarily as supporting elements. Collagen generally includes segments of triple-helical conformation and possesses the capacity for self-assembly into extracellular aggregates.

The major physiological functions of collagen are accomplished by extracellular aggregates of the molecules, and the structure of the aggregates is directly related to specific function. Several unique modes of aggregation have been discerned. For example, one of the most prevalent type of aggregate is the fiber, and the capacity to form fibers is common to many types of collagens. In general, fiber formation involves lateral association and axial displacement of molecules arranged in parallel. Bundled fibers lead to body tissue formation. Other known modes of aggregation involve associations of molecules allowing some degree of antiparallel orientation of individual molecules.

As understood by those skilled in the art, the collagenous scaffold of the extracellular matrix includes at least 13, and up to 20, genetically distinct types of collagen. The most abundant and well characterized collagen is referred to as type 1 by those of skill in the art, and may be extracted from bovine (cow) hide. Other sources include porcine (pig), porcine hide, equine (horse), equine hide, chicken tendon, bovine tendon, and various others. Collagen types 1, 3, and 5 are typically specific for skin. Comprehensive reviews of the role of the collagenous matrix in organ-specific tissue repair, as understood by those skilled in the art, illustrate the advantageous role collagens play in wound healing and tissue repair.

### SUMMARY OF THE INVENTION

The present invention provides for a method and apparatus for treating a wound. In a preferred method, collagen is applied to a wound; a drain tube is placed adjacent the wound, and a wound dressing is applied over the collagen and the drain tube. The preferred method further includes the step of draining fluids from the wound through the drain tube, which tube has a proximal end positioned adjacent the wound and a distal end in fluid communication with a vacuum source. The fluids are drained from the wound by applying a negative pressure to the wound through the tube from the vacuum source.

### BRIEF DESCRIPTION OF THE DRAWINGS

FIG. 1 is a cross-sectional view of a wound treatment apparatus in accordance with the present invention.

FIG. 2 is a top view of the wound treatment apparatus of FIG. 1.

FIG. 3 is a cross-sectional view of a wound treatment apparatus in accordance with the present invention.

FIG. 4 is a top view of the wound treatment apparatus of FIG. 3.

FIG. 5 is a flow diagram illustrating an embodiment of a method in accordance with the present invention.

FIG. 6 is a flow diagram illustrating a further embodiment of a method in accordance with the present invention.

### DETAILED DESCRIPTION

Although the following detailed description contains many specific details for purposes of illustration, anyone of ordinary skill in the art will appreciate that many variations and alterations to the following details are within the scope of the invention. Accordingly, the exemplary embodiment of the invention described below is set forth without any loss of generality to, and without imposing limitations thereon, the claimed invention.

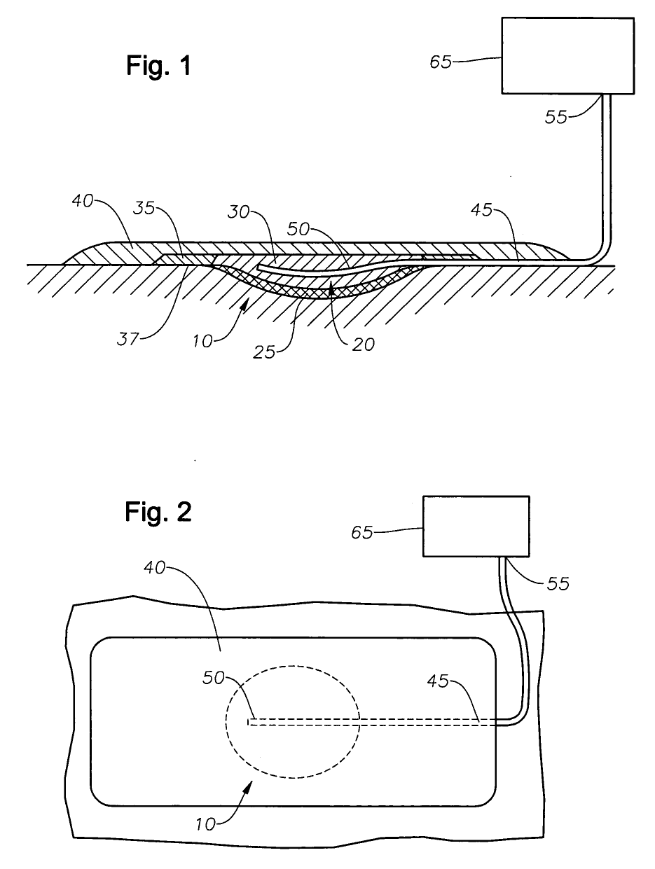

Referring to FIG. 1, a wound 10 is illustrated on a part of a body. Wound 10 can be, for example, a cut, scrape, scratch, sore, blister, incision, gouge, abrasion, or other type of wound. A first step in treating wound 10 preferably is to cleanse the wound gently and carefully with a saline solution, a soap and water solution, a sterile water solution, or other non-toxic solution to the extent that this initial cleaning procedure is helpful to remove debris, necrotic slough, or other material from wound 10. The water pressure during this cleansing typically should not exceed 8 pounds per square inch. After the cleansing procedure, wound 10 preferably should be blotted dry by a sanitary towel or other material.

Collagen 25 preferably is applied next to wound 10. The most preferred types of collagen 25 used in accordance with a method of the present invention are porcine collagen and bovine collagen; however, each of the genetically distinct types of collagen alternatively can be used in accordance with the present invention.

Collagen 25 is typically applied in a granular or particulate form, but also can be applied in other forms, such as, for example, a moist slurry of collagen particles. Collagen 25 preferably is applied as a relatively thin layer to a wound bed surface of wound 10, such as, for example, a layer of collagen particles about 1/16 inch thick that substantially covers the wound bed surface of wound 10. Collagen 25 preferably is not layered thickly and is not packed tightly onto the wound bed surface of wound 10. Thicker layers can be applied to treat more severe wounds that feature heavy drainage or infection. These more severe wounds may require a thicker layer of collagen 25 up to about ⅛ inch thick. In more severe wounds where drainage has slowed or infection diminishes, subsequent applications of collagen 25 may form a relatively thin layer of collagen 25 applied to the wound bed surface of wound 10. Therefore, generally, more collagen 25 is applied to heavily draining wounds, and less collagen 25 is applied to lightly draining wounds.

In an alternative embodiment, a topical medication may be applied to the wound bed surface of wound 10 before application of collagen 25. In this embodiment, it is preferable to apply a relatively thicker layer of collagen 25 that substantially covers the topical medication and wound bed surface of wound 10.

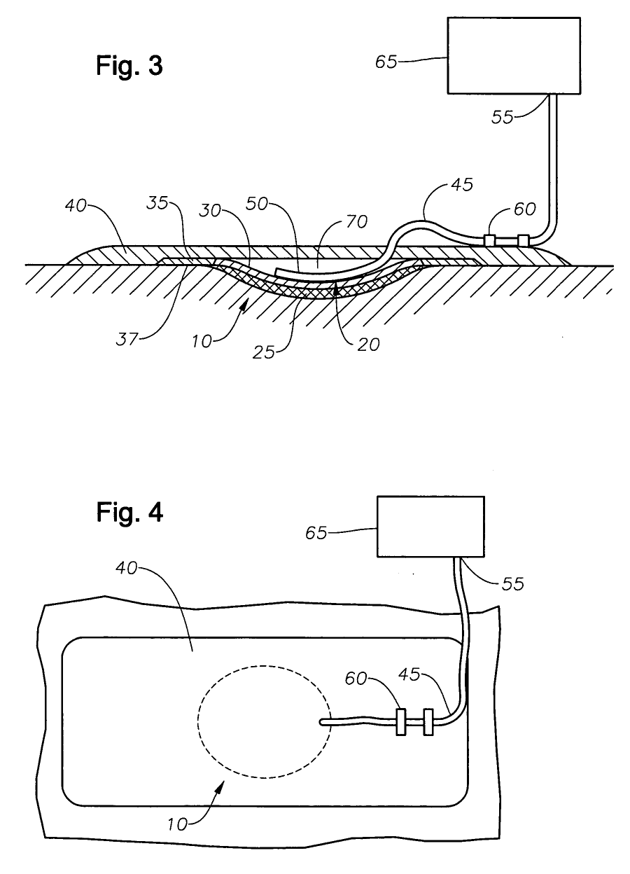

After collagen 25 is applied to wound 10, gauze 30 preferably is positioned overlying collagen 25 on wound 10, as best illustrated in FIG. 3. Gauze 30 preferably is anti-microbial to prevent infection and advance the healing of wound 10. Additional gauze preferably may be fluffed, and preferably moistened with saline, to cover proximal end 50 of drain tube 45 with gauze and fill space 70 with gauze to skin level.

After gauze 30 is positioned overlaying collagen 25 on wound 10, a protective cream or ointment 35 preferably can be applied on peri-wound margin 37 of wound 10 adjacent collagen 25 and gauze 30 to thereby reduce risk of infection and provide additional skin protection. Alternatively, protective cream or ointment 35 may be applied before gauze 30 is positioned overlying collagen 25 on wound 10 or after drain tube 45 is positioned adjacent wound 10. Peri-wound margin 37 of wound 10 is the area immediately surrounding wound 10 that extends, for example, from the wound edge to about one inch or more onto the intact skin from the wound edge. The distance a peri-wound margin extends often depends upon the size and depth of the wound. Epithelization, one of the wound healing phases, originates from the wound edges. Protective cream or ointment 35 protects peri-wound margin 37 and the wound edges from damage and maceration, enabling the epithelization to proceed properly.

After collagen 25, gauze 30, and protective cream or ointment 35 are applied to wound 10, proximal end 50 of drain tube 45 preferably is positioned on top of gauze 30, as best illustrated in FIG. 3. More preferably gauze 30 is wrapped around proximal end 50 of drain tube 45 such that any loose threads of gauze 30 are wrapped inward, as best illustrated in FIG. 1. This arrangement helps to prevent loose threads of gauze 30 from adhering to wound 10 during the wound healing process. In either embodiment, gauze 30 helps to prevent proximal end 50 of drain tube 45 from becoming clogged. Proximal end 50 of drain tube 45 is preferably a Jackson-Pratt or channel drain, as understood by those of skill in the art.

Drain tube 45 is utilized to drain fluids 20 from wound 10 to augment the healing process. Proximal end 50 of drain tube 45 extends adjacent to wound 10, and distal end 55 of drain tube 45 extends away from wound 10.

In a preferred embodiment best illustrated in FIGS. 1 and 2, semi-permeable dressing 40 is applied over wound 10, collagen 25, gauze 30, protective cream or ointment 35, and proximal end 50 of drain tube 45. A semi-permeable wound dressing is preferred in order to increase patient comfort and lower the risk of infection. Semi-permeable dressings generally allow gases to pass therethrough thus permitting a wound beneath the dressing to breath, yet are generally impervious or impermeable to liquids. Semi-permeable dressing 40 preferably covers the entire surface area of wound 10 and extends onto and preferably beyond peri-wound margin 37. Semi-permeable dressing 40 preferably includes an adhesive capable of creating a generally air-tight seal over the layers below and holding them in a substantially fixed position. Semi-permeable dressing 40 is preferably an OPSITE® semi-permeable dressing, available from Smith & Nephew Medical, Ltd., but can be any suitable semi-permeable dressing. Furthermore, semi-permeable dressing 40 can be made of an anti-microbial material to prevent infection and advance wound healing. In this embodiment, drain tube 45 extends out from under an edge of semi-permeable dressing 40. Adhesive pastes or tapes can be used to ensure an airtight seal where the dressing meets drain tube 45. In a particularly preferred embodiment, one end of semi-permeable dressing 40 is split in order to form arms that wrap around drain tube 45.

In an alternative embodiment best illustrated in FIGS. 3 and 4, semi-permeable dressing 40 can include an aperture or opening that facilitates insertion of drain tube 45 therethrough. The aperture or opening may be created in semi-permeable dressing 40 either before or after semi-permeable dressing 45 is applied over wound 10. The proximal end 50 of drain tube 45 is inserted through the aperture or opening in semi-permeable dressing 40 to a position adjacent wound 10 and above the layer of collagen 25, such that proximal end 50 of drain tube 45 is positioned properly to drain fluids 20 from wound 10. Proximal end 50 of drain tube 45 preferably should be positioned on a layer of gauze 30 or wrapped in gauze 30 to prevent clogging.

It also is possible to use a piece of gauze as a dressing in place of semi-permeable dressing 40. In that case, although drainage of fluids can occur through drain tube 45, less negative pressure will be created in the vicinity of the wound. A cloth or compression bandage optionally may be placed over semi-permeable dressing 40 if additional support is required to hold the apparatus in place.

Distal end 55 of drain tube 45 extends away from semi-permeable dressing 40 to vacuum source 65, such as, for example, a vacuum pump or suction pump. Preferred vacuum sources include the Vario and the Dominant 35c/i, both of which are commercially available from Medela AG. Vacuum pumps such as these can be used for a variety of suctioning procedures in addition to drainage of fluids from wounds, such as, for example, nasopharyngeal, tracheal, surgical, gastrointestinal, and thoracic drainage, as understood by those skilled in the art. As best illustrated in FIGS. 3 and 4, portions of drain tube 45 that extend outward from semi-permeable dressing 40 can be affixed or fastened to semi-permeable dressing 40 by fastener 60 to stabilize proximal end 50 of drain tube 45 in a substantially fixed position adjacent wound 10. Preferably after portions of drain tube 45 are affixed to semi-permeable dressing 40, distal end 55 of drain tube 45 is placed in fluid communication with vacuum source 65.

After proximal end 50 of drain tube 45 is positioned adjacent wound 10 as previously described and semi-permeable dressing 40 is sealed over wound 10, vacuum source 65 is activated. Vacuum source 65 applies a negative pressure or suction to wound 10 through drain tube 45, thereby draining fluids 20 from wound 10 through drain tube 45 and into vacuum source 65. Drainage of fluids 20 from wound 10 is beneficial to the healing process. Embodiments of the invention can provide suction of many wound drainage fluids 20 under various circumstances, such as surgical fluids, bodily fluids, gases, tissue, infectious materials from wounds, viscous puss-like fluids including white blood cells, cellular debris, and necrotic tissue, and other drained fluids, as understood by those skilled in the art. The negative pressure or vacuum preferably is high enough to facilitate flow of wound drainage fluids 20 through drain tube 45, but not so high that the negative pressure or vacuum disrupts collagen 25 or damages the healing structure of the wound itself, as understood by those of skill in the art. In a preferred embodiment, the application of negative pressure is stopped and a new wound treatment apparatus is applied to the wound as described above approximately every two days until the wound has healed or the wound treatment is otherwise ceased.

Embodiments of the present invention offer important advantages and benefits. Application of embodiments of the invention complies with and exceeds all applicable standards of care for medical or health care professionals, and complies with and exceeds all applicable regulatory requirements. Embodiments of the invention advantageously combine the beneficial use of collagen with the draining of fluids from a wound, while allowing the body's natural wound healing processes to function properly, which processes include the complex sequential cellular activities of the wound healing process necessary for wound closure. Embodiments of the invention advantageously establish and maintain a moist, clean, and biologically active wound environment to sustain sequential cellular activities. Embodiments of the invention also manage and control wound bed interference and such properties as improper moisture, inadequate oxygen, infection from bacteria, and other factors that affect the wound healing process. Application of embodiments of the invention advantageously enhances reduction of infection in chronic, acute, traumatic, subacute, and dehisced wounds, diabetic ulcers, pressure ulcers, flaps, grafts, and partial thickness burns.

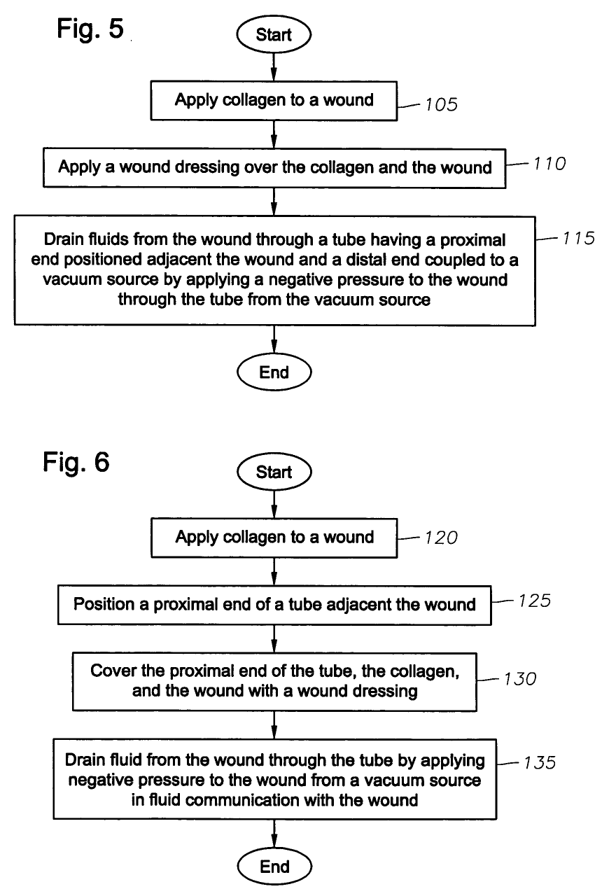

FIG. 5 is a flow diagram illustrating a preferred embodiment of a method in accordance with the present invention. Collagen is applied to a wound in step 105. A wound dressing is applied over the collagen and the wound in step 110. Fluids then are drained from the wound through a tube having a proximal end positioned adjacent the wound and a distal end coupled to a vacuum source by applying a negative pressure to the wound through the tube from the vacuum source in step 115.

FIG. 6 is a flow diagram illustrating a further preferred embodiment of a method in accordance with the present invention. Collagen is applied to a wound in step 120. A proximal end of a tube is positioned adjacent the wound in step 125. The proximal end of the tube, the collagen, and the wound are covered with a wound dressing in step 130. Fluids then are drained from the wound through a tube by applying negative pressure to the wound from a vacuum source in fluid communication with the wound in step 135.

Although some embodiments of the present invention have been described in detail, it should be understood that various changes, substitutions, and alterations can be made hereupon without departing from the principle and scope of the invention. Accordingly, the scope of the present invention should be determined by the following claims and their appropriate legal equivalents.

That claimed is:

1. A method for treating a wound, comprising:
(a) applying collagen to the wound;
(b) applying a wound dressing over the collagen and the wound; and
(c) draining fluids from the wound through a tube having a proximal end positioned adjacent the wound and a distal end coupled to a vacuum source by applying a negative pressure to the wound through the tube from the vacuum source.

2. The method according to claim 1, further comprising the step of positioning a gauze over the collagen.

3. The method according to claim 1, further comprising the step of wrapping the proximate end of the tube in gauze.

4. The method according to claim 1, wherein the wound dressing comprises a semi-permeable dressing.

5. The method according to claim 1, wherein step (c) further comprises extending the proximal end of the tube through an aperture in the wound dressing to a position adjacent the wound before draining fluids from the wound.

6. The method according to claim 1, further comprising the step of applying a protective cream on a peri-wound margin.

7. The method according to claim 1, wherein the collagen is applied in a layer about 1/16 to ⅛ inch thick.

8. The method according to claim 1, wherein step (a) comprises applying a topical medication to the wound, and thereafter applying a layer of collagen that substantially covers the topical medication.

9. A method for dressing a wound, comprising:
(a) applying collagen to the wound;
(b) positioning a proximal end of a tube adjacent the wound;
(c) covering the proximal end of the tube, the collagen, and the wound with a wound dressing; and
(d) draining fluid from the wound through the tube by applying negative pressure to the wound from a vacuum source in fluid communication with the wound.

10. The method according to claim 9, wherein the wound dressing comprises a semi-permeable dressing.

11. The method according to claim 9, further comprising the step of applying a protective cream on a peri-wound margin.

12. The method according to claim 9, further comprising the step of positioning a gauze over the collagen.

13. The method according to claim 9, further comprising the step of wrapping the proximate end of the tube in gauze.

14. An apparatus for treating a wound, comprising:
collagen;
a wound dressing for covering the collagen and the wound; and
a vacuum source for evacuating fluids from the wound.

15. The apparatus according to claim 14, further comprising a topical medication for application to the wound, wherein the collagen is applied in covering relation to the topical medication.

16. The apparatus according to claim 14, further comprising a gauze for overlying the collagen, the gauze adapted to be covered by the wound dressing.

17. The apparatus according to claim 14, wherein the wound dressing comprises a semi-permeable dressing.

18. The apparatus according to claim 14, further comprising a tube having a proximal end for placement adjacent the wound and a distal end connected to the vacuum source.

19. The apparatus according to claim 18, wherein the tube extends under an edge of the wound dressing.

20. The apparatus according to claim 18, wherein the tube extends through an aperture in the wound dressing.

21. The apparatus according to claim 18, further comprising a fastener for affixing the tube to the wound dressing to stabilize the proximal end of the tube in a substantially fixed position adjacent the wound.

22. The apparatus according to claim 18, further comprising a gauze wrapped around the proximate end of the tube.
